(19) United States
(12) Patent Application Publication
(10) Pub. No.: US 2005/0090668 A1
(43) Pub. Date: Apr. 28, 2005

(54) Preparation of 5-hydroxy-6-oxo-1,6-dihydropyrimidine compounds

(76) Inventor: Spencer D. Dreher, Metuchen, NJ (US)

Correspondence Address: Kenneth R. Walton;Merck & Co., Inc., Patent Dept., RY60-30, P.O. Box 2000, Rahway, NJ, 07065-0907, US

(21) Appl. No.: 10/954,752

(22) Filed: Sep. 30, 2004

Related U.S. Application Data
(60) Provisional application No. 60/514,880, filed on Oct. 28, 2003.

(51) Int. Cl. C07D 239/02

(52) U.S. Cl. 544/309

(57) ABSTRACT

5-Hydroxy-6-oxo-1,6-dihydropyrimidine compounds are prepared by the condensation of dihydroxyfumarate derivatives with amidines. The pyrimidine compounds are useful as intermediates in the preparation of pharmacologically active compounds.

This application claims the benefit of U.S. Provisional Application No. 60/514,880, filed Oct. 28, 2003, the disclosure of which is hereby incorporated by reference in its entirety.

### FIELD OF THE INVENTION

The present invention is directed to a process for preparing 5-hydroxy-6-oxo-1,6-dihydropyrimidine compounds and to a class of dioxyfumarate reagents that can be employed as reactants in the process. The 5-hydroxy-6-oxo-1,6-dihydropyrimidines are useful as intermediates in the preparation of pharmacologically active compounds.

### BACKGROUND OF THE INVENTION

Pyrimidines are useful because of their pharmacological activity. For example, the pyrimidine carboxamides described in WO 03/035076 and in WO 03/035077 are HIV integrase inhibitors that can be used to treat HIV infection and AIDS. 2-Substituted-5-hydroxy-6-oxo-1,6-dihydropyrimidine carboxylic acid derivatives are useful as intermediates in the preparation of pyrimidine carboxamides, but no simple, high-yielding method for preparing these derivatives has been developed. One well-known route involves the Michael reaction of N-hydroxy amidines and acetylynic diesters, followed by a thermal Claisen rearrangement and amide condensation. Overall yields for this route are typically in the 30 to 40% range. Another route involves the condensation of certain dihydroxyfumarate derivatives with amidines, but yields via this route are typically reported to be low.

The present invention is directed to an improved method for preparing pyrimidines using novel dihydroxyfumarate derivatives.

The following references are of interest as background:

Johnson et al., J. Am. Chem. Soc. 1929, 51: 873-880 discloses the preparation of 2-ethylmercapto-4-ethylcarboxylate-5-ethoxy-6-oxopyrimidine by first reacting ethyl oxalate with ethyl ethoxyacetate in anhydrous ether in the presence of Na metal, and then adding pseudo-ethylthiourea hydrobromide.

Budesinsky et al., J. Collect. Czech. Chem. Commun. 1962, 27: 2550-2560 discloses the preparation of 5-chloro-2-methyl-4-oxo-6-ethylcarboxylate by first reacting ethyl oxalate with ethyl chloroacetate, and then adding acetamidine HCl.

Jaffe et al. J. Org. Chem. 1968, 33: 4004-4010 discloses the preparation of dimethyl dihydroxy fumarate by the esterification of dihydroxyfumaric acid with methanol in the presence of anhydrous magnesium sulfate and anhydrous HCl.

Bender et al., J. Org. Chem. 1978, 43: 3354-3362 discloses the preparation of 2-methoxy-3-hydroxy fumarate ethyl ester t-butyl ester by treating t-butyl methoxyacetate with Li cycloohexylisopropylamide, followed by condensation with diethyl oxalate.

Culbertson, J. Heterocycl. Chem. 1979, 16: 1423-1424 discloses the prepartion of the methyl ester of 5,6-dihydroxy-2-phenyl-4-pyrimidinecarboxylic acid by refluxing dimethylacetylenedicarboxylate with benzamide oxime and then heating the resulting adduct to cause cyclization. A similar disclosure is found in Cooper et al., J. Chem. Soc. Perkins Trans. 1 1976, 2038-2045.

Sunderland et al., Inorg. Chem. 2001, 40: 6746-6756 discloses the preparation of 2-methyl-3H-5-benzyloxy-6-carboxy-4-pyrimdinone ethyl ester by first reacting benzyl benzyloxyacetate and diethyloxalate in dry THF with NaH to obtain 2-benzyloxy-3-hydroxy fumarate ethyl ester benzyl ester and then reacting the diester with acetamidine HCl and sodium ethoxide.

“The Principle Synthetic Method”, Chapter 2 (pp. 31-81) in D. J. Brown, The Pyrimidines (John Wiley, New York, 1962) discloses the preparation of various pyrimidines via the condensation of amidines with various diketo compounds.

### SUMMARY OF THE INVENTION

The present invention is directed to processes for preparing 5-hydroxy-6-oxo-1,6-dihydropyrimidines. More particularly, the present invention includes a process for preparing a compound of Formula I: 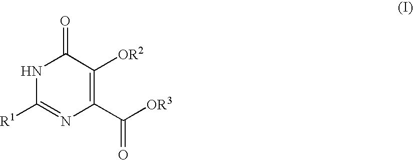
which comprises reacting an amidine of Formula II, or an acid salt thereof: 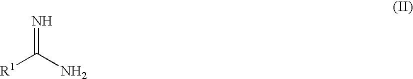
with a dioxyfumarate of Formula III: 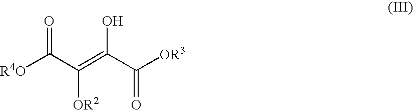
in an organic solvent and in the presence of a base to obtain the compound of Formula I; wherein:
R¹ is:
   (1) —H,
   (2) —O—C₁₋₆ alkyl,
   (3) —CO₂R^{a},
   (4) —C₁₋₆ alkyl,
   (5) —C₁₋₆ alkylene-CO₂R^{a},
   (6) —C₁₋₆ alkylene-N(R^{b}R^{c}),
   (7) —C₃₋₈ cycloalkyl, which is optionally substituted with from 1 to 4 substituents each of which is independently —OH, —CN, or —C₁₋₆ alkyl,
   (8) —C₁₋₆ alkylene-C₃₋₈ cycloalkyl, where the cycloalkyl is optionally substituted with from 1 to 4 substituents each of which is independently —OH, —CN, or —C₁₋₆ alkyl,
   (9) aryl, which is optionally substituted with from 1 to 5 substituents each of which is independently halo, —OH, —CN, —NO₂, —CO₂R^{a}, —C₁₋₆ alkyl, —O—C₁₋₆ alkyl, —C₁₋₆ alkylene-OH, or —C₁₋₆ alkylene-O—C₁₋₆ alkyl,
   (10) —C₁₋₆ alkylene-aryl, where the aryl is optionally substituted with from 1 to 5 substituents each of which is independently halo, —OH, —CN, —NO₂, —CO₂R^{a}, —C₁₋₆ alkyl, —O—C₁₋₆ alkyl, —C₁₋₆ alkylene-OH, or —C₁₋₆ alkylene-O—C₁₋₁₆ alkyl,
   (11) heterocycle, which is (i) optionally substituted with from 1 to 4 substituents each of which is independently halo or a —C₁₋₆ alkyl, and (ii) optionally mono-substituted with aryl or —C₁₋₆ alkylene-aryl, HetC or —C₁₋₁₆ alkylene-HetC, or
   (12) —C₁₋₆ alkylene-heterocycle, where the heterocycle is (i) optionally substituted with from 1 to 4 substituents each of which is independently halo or a —C₁₋₆ alkyl, and (ii) mono-substituted with aryl or —C₁₋₆ alkylene-aryl, HetC or —C₁₋₆ alkylene-HetC;
R² is:
   (1) —C₁₋₆ alkyl,
   (2) aryl, which is optionally substituted with from 1 to 5 substituents each of which is independently halo, —OH, —CN, —NO₂, —CO₂R^{a}, —C₁₋₆ alkyl, —O—C₁₋₆ alkyl, —C₁₋₆ alkylene-OH, or —C₁₋₆ alkylene-O—C₁₋₆ alkyl, or
   (3) —C₁₋₆ alkylene-aryl, where the aryl is optionally substituted with from 1 to 5 substituents each of which is independently halo, —OH, —CN, —NO₂, —CO₂R^{a}, —C₁₋₆ alkyl, —O—C₁₋₆ alkyl, —C₁₋₆ alkylene-OH, or —C₁₋₆ alkylene-O—C₁₋₆ alkyl;
R³ is —C₁₋₆ alkyl;
R⁴ is methyl or ethyl;
   each R^{a} is independently —H or —C₁₋₆ alkyl;
R^{b} is:
   (1) —C₁₋₆ alkyl,
   (2) aryl, which is optionally substituted with from 1 to 4 substituents each of which is independently halo, —OH, —CN, —NO₂, —C₁₋₆ alkyl, or —O—C₁₋₆ alkyl, or
   (3) P^{b}, which is an amine protective group;
R^{c} is:
   (1) —C₁₋₆ alkyl,
   (2) aryl, which is optionally substituted with from 1 to 4 substituents each of which is independently halo, —OH, —CN, —NO₂, —C₁₋₆ alkyl, or —O—C₁₋₁₆ alkyl, or
   (3) P^{c}, which is an amine protective group;
      each aryl is independently phenyl or naphthyl;
      heterocycle is (i) a 4- to 8-membered, saturated or unsaturated monocyclic ring, (ii) a 7- to 12-membered bicyclic ring system, or (iii) an 11 to 16-membered tricyclic ring system; wherein each ring in (ii) or (iii) is independent of, bridged with, or fused to the other ring or rings and each ring is saturated or unsaturated, and the monocyclic ring, bicyclic ring system, or tricyclic ring system contains at least one carbon atom and from 1 to 6 heteroatoms independently selected from N, O and S; wherein any one or more of the nitrogen and sulfur heteroatoms is optionally oxidized; and
      HetC is (i) a 5- or 6-membered heteroaromatic ring containing from 1 to 4 heteroatoms independently selected from N, O and S or (ii) a 4- to 7-membered saturated heterocyclic ring containing at least one carbon atom and from 1 to 4 heteroatoms independently selected from N, O and S; wherein any one or more of the nitrogen and sulfur heteroatoms in (ii) is optionally oxidized.

The process of the present invention can provide the pyrimidine compounds of Formula I in high yield. Various embodiments, aspects and features of the present invention are either described in or will be apparent from the ensuing description, examples, and appended claims.

### DETAILED DESCRIPTION OF THE INVENTION

A compound of Formula I is alternatively referred to herein more simply as “Compound I”. Similarly, an amidine of Formula II is alternatively referred to as “amidine II” or “Compound II”, and a dioxyfumarate of Formula III is alternatively referred to as “dioxyfumarate III” or “Compound III”. Analogous nomenclature is employed for compounds of Formula IV, V-A, V-B, VI, VII-A, and VII-B presented in the description below.

The present invention includes processes for preparing 2-substituted-5-hydroxy-6-oxo-1,6-dihydropyrimdine-4-carboxylic acid derivatives via the condensation of dioxyfumarates with amidines. This process is set forth above in the Summary of the Invention.

A first embodiment of the process of the invention is the process as set forth above, wherein R¹ is:
(1) —H,
(2) —O—C₁₋₄ alkyl,
(3) —CO₂R^{a},
(4)-C₁₋₄ alkyl,
(5) —C₁₋₄ alkylene-CO₂R^{a},
(6) —C₁₋₄ alkylene-N(R^{b}R^{c}),
(7)—C₃₋₆ cycloalkyl, which is optionally substituted with from 1 to 3 substituents each of which is independently a —C₁₋₄ alkyl,
(8) —C₁₋₄ alkylene-C₃₋₆ cycloalkyl, where the cycloalkyl is optionally substituted with from 1 to 3 substituents each of which is independently a —C₁₋₄ alkyl,
(9)-phenyl, which is optionally substituted with from 1 to 3 substituents each of which is independently halo, —CN, —NO₂, —CO₂R^{a}, —C₁₋₄ alkyl, or —O—C₁₋₄ alkyl,
(10) —C₁₋₄ alkylene-phenyl, where the phenyl is optionally substituted with from 1 to 3 substituents each of which is independently halo, —CN, —NO₂, —CO₂R^{a}, —C₁₋₄ alkyl, or —O—C₁₋₄ alkyl,
(11)-HetA,
(12) —C₁₋₄ alkylene-HetA,
(13)-HetB, or
(14) —C₁₋₄ alkylene-HetB;
   each R^{a} is independently —H or —C₁₋₄ alkyl;
R^{b} is:
   (1) —C₁₋₄ alkyl (e.g., methyl or ethyl, or methyl only),
   (2) phenyl, which is optionally substituted with from 1 to 3 substituents each of which is independently halo, —NO₂, —C₁₋₄ alkyl, or —O—C₁₋₄ alkyl, or
   (3) P^{b} which is an amine protective group selected from the group consisting of:
   (i) —CH₂-phenyl, where the phenyl is optionally substituted with from 1 to 3 substituents each of which is independently halo, —NO₂, —C₁₋₄ alkyl, or —O—C₁₋₄ alkyl,
   (ii)-C(═O)—C₁₋₄ alkyl,
   (iii)-C(═O)—CH₂-phenyl,
   (iv)-C(═O)—O—C₁₋₄ alkyl,
   (v) —C(═O)—O—CH₂—CH═CH₂, or
   (vi)-C(═O)—O—CH₂-phenyl, where the phenyl is optionally substituted with from 1 to 3 substituents each of which is independently halo, —NO₂, —C₁₋₄ alkyl, or —O—C₁₋₄ alkyl;
R^{c} is:
   (1) —C₁₋₄ alkyl (e.g., methyl or ethyl, or methyl only),
   (2) phenyl, which is optionally substituted with from 1 to 3 substituents each of which is independently halo, —NO₂, —C₁₋₄ alkyl, or —O—C₁₋₄ alkyl, or
   (3) P^{c} which is an amine protective group that independently has the same definition as P^{b} in the definition of R^{b} above;
      HetA is a 5- or 6-membered heteroaromatic ring containing from 1 to 4 heteroatoms independently selected from N, O and S; wherein the heteroaromatic ring is (i) optionally substituted with from 1 to 3 substituents each of which is independently halo or a —C₁₋₄ alkyl and (ii) optionally mono-substituted with phenyl or —(CH₂)₁₋₂-phenyl;
      HetB is a 4- to 7-membered saturated heterocyclic ring containing at least one carbon atom and a total of from 1 to 4 heteroatoms independently selected from N, O and S; wherein any ring sulfur is optionally substituted with oxo or dioxo; and wherein the heterocyclic ring is (i) optionally substituted with from 1 to 3 substituents each of which is independently halo or a —C₁₋₄ alkyl and (ii) optionally mono-substituted with phenyl, —(CH₂)₁₋₂-phenyl, HetC, or —(CH₂)₁₋₂-phenyl-HetC; and
HetC is (i) a 5- or 6-membered heteroaromatic ring containing from 1 to 4 heteroatoms independently selected from N, O and S or (ii) a 4- to 7-membered saturated heterocyclic ring containing at least one carbon atom and from 1 to 4 heteroatoms independently selected from N, O and S; wherein any one or more of the nitrogen and sulfur heteroatoms in (ii) is optionally oxidized.

In the original definition of R¹ (i.e., its definition in the Summary of the Invention), —C₁₋₆ alkylene-N(R^{b} R^{c}) can be a protected aminoalkyl group; i.e., either or both of R^{b} and R^{c} can be the same or different amine protective groups either or both of which can under certain conditions be removed (cleaved) to provide either a primary or a secondary amine. Thus, a second embodiment of the process of the invention is the process as originally described, wherein, when R¹ in Compound I is —C₁₋₆ alkylene-N(R^{b}R^{c}) in which (a) R^{b} is P^{b} and R^{c} is P^{c} or (b) R^{b} is P^{b} and R^{c} is other than P^{c} (i.e., R^{c} is —C₁₋₆ alkyl or aryl optionally substituted with from 1 to 4 substituents each of which is independently halo, —OH, —CN, —NO₂, —C₁₋₆ alkyl, or —O—C₁₋₆ alkyl), the process optionally further comprises treating Compound I with one or more amine deprotecting agents (concurrently or sequentially) to obtain a primary (—NH₂) or a secondary amine (—NHR^{c}) derivative thereof. Suitable amine protective groups and agents and conditions for their removal are well known in the art and include, for example, those described in Greene and Wuts, Protective Groups in Organic Synthesis, 2d edition, (Wiley-Interscience, 1991), pp. 309-405 (herein incorporated by reference); and in McOmie, Protective Groups in Organic Synthesis (Plenum, 1973), pp. 44-74 (herein incorporated by reference). In an aspect of the second embodiment of the process of the invention, when R¹ is —C₁₋₄ alkylene-N(R^{b}R^{c}), R^{b} is P^{b} (i.e., R^{b} is (i)-CH₂-phenyl, where the phenyl is optionally substituted with from 1 to 3 substituents each of which is independently halo, —NO₂, —C₁₋₄ alkyl, or —O—C₁₋₄ alkyl, (ii) —C(═O)—C₁₋₄ alkyl, (iii)-C(═O)—CH₂-phenyl, (iv)-C(═O)—O—C₁₋₄ alkyl, (v) —C(═O)—O—CH₂—CH═CH₂, or (vi)-C(═O)—O—CH₂-phenyl, where the phenyl is optionally substituted with from 1 to 3 substituents each of which is independently halo, —NO₂, —C₁₋₄ alkyl, or —O—C₁₋₄ alkyl), and R^{c} is —C₁₋₄ alkyl, then the process optionally further comprises treating Compound I with an amine deprotecting agent to remove (i.e., cleave) P^{b} and obtain thereby the following derivative of Compound I which is a secondary amine of Formula IV: 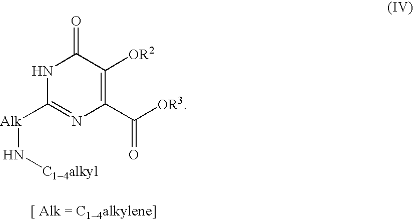
In another aspect of the second embodiment of the process, suitable P^{b} and P^{c} groups are each independently a —C(═O)—O—R* group, wherein R* is t-butyl (i.e., R^{b} and/or R^{c}=Boc), benzyl (Cbz), allyl (Alloc), p-nitrobenzyl, p-methoxybenzyl, p-bromobenzyl, or p-chlorobenzyl.

As already noted above, methods for removing the protective groups P^{b} and/or P^{c} are well known in the art. In many instances the P^{b} and/or P^{c} (e.g., alkyloxycarbonyl groups such as Boc, alkylcarbonyl groups such as acetyl, and others) can be removed by treatment with acids including mineral acids, Lewis acids, or organic acids. Suitable mineral acids include hydrogen halides (HCl, HBr, and HF, as a gas or in aqueous solution), sulfuric acid, and nitric acid. Suitable organic acids include carboxylic acids, alkylsulfonic acids and arylsulfonic acids. Exemplary organic acids include trifluoroacetic acid, toluenesulfonic acid, benzenesulfonic acid, methanesulfonic acid, and trifluoromethanesulfonic acid. Suitable Lewis acids include BF₃.Et₂O, SnCl₄, ZnBr₂, Me₃SiI, Me₃SiCl, Me₃SiOTf, and AlCl₃. Cleavage conditions (e.g., temperature, choice and concentration of acid) can vary from mild to harsh depending upon the lability of the amino protective group. The selection of appropriate conditions can be determined without undue experimentation by a person of ordinary skill in the art. Although acid treatment is often effective, other means can be employed. Removal of Cbz or Alloc, for example, is typically accomplished via hydrogenolysis (e.g., hydrogenation with a Pd catalyst). Reference is made to Greene and Wuts and to McOmie (both cited above) for further details.

In some cases, another functional group in the molecule (e.g., —C(═O)OR³) is unstable or reactive under the conditions selected for removing protective groups P^{b} and/or P^{c}. Thus, in another aspect of the second embodiment of the invention, the process further comprises protecting a functional group in Compound I that is unstable or reactive under the conditions employed to remove P^{b} and/or P^{c}, and then deprotecting the functional group subsequent to the removal of P^{b} and/or P^{c}. Reference is made to Greene and Wuts and to McOmie (both cited above) for further details on the protection and deprotection of various functional groups.

A third embodiment of the process of the invention is the process as originally set forth above, wherein R¹ is:
(1) —H,
(2) —C₁₋₄ alkyl,
(3) cyclopropyl,
(4) —CH₂-cyclopropyl,
(5) phenyl, which is optionally substituted with from 1 to 3 substituents each of which is independently chloro, bromo, fluoro, —CN, —NO₂, —C₁₋₄ alkyl, or —O—C₁₋₄ alkyl,
(6) —CH₂-phenyl, where the phenyl is optionally substituted with from 1 to 3 substituents each of which is independently chloro, bromo, fluoro, —CN, —NO₂, —C₁₋₄ alkyl, or —O—C₁₋₄ alkyl,
(7) -HetA,
(8) —CH₂-HetA,
(9) -HetB, or
(10) —CH₂-HetB;
   HetA is a heteroaromatic ring selected from the group consisting of pyrrolyl, pyrazolyl, imidazolyl, triazolyl, tetrazolyl, thiophenyl, thiazolyl, isothiazolyl, oxazolyl, isoxazolyl, oxadiazolyl, pyridinyl, pyridazinyl, pyrimidinyl, and pyrazinyl; wherein the heteroaromatic ring is optionally mono-substituted with a —C₁₋₄ alkyl, phenyl, or benzyl; and
   HetB is a saturated heterocylic ring selected from the group consisting of pyrrolidinyl, pyrazolidinyl, imidazolidinyl, thiazolidinyl, isothiazolidinyl, oxazolidinyl, isoxazolidinyl, piperidinyl, piperazinyl, morpholinyl, and thiomorpholinyl; wherein the heterocyclic ring is optionally mono-substituted with a —C₁₋₄ alkyl, phenyl, or benzyl.

A fourth embodiment of the process of the invention is the process as originally described, wherein R² is:
(1) —C₁₋₆ alkyl (e.g., —C₁₋₄ alkyl),
(2) phenyl, which is optionally substituted with from 1 to 3 substituents each of which is independently halo, —CN, —NO₂, —CO₂H, —C(═O)—O—C₁₋₃ alkyl, —C₁₋₁₄ alkyl, or —O—C₁₋₄ alkyl, or
(3) —CH₂-phenyl, where the phenyl is optionally substituted with from 1 to 5 substituents each of which is independently halo, —CN, —NO₂, —CO₂H, —C(═O)—O—C₁₋₃ alkyl, —C₁₋₄ alkyl, or —O—C₁₋₄ alkyl;
   and all other variables are as originally defined or as defined in any one of the preceding embodiments.

A fifth embodiment of the process of the invention is the process as originally described, wherein R² is benzyl; and all other variables are as originally defined or as defined in any one of the first three embodiments.

A sixth embodiment of the process of the invention is the process as originally described, wherein R³ is a branched —C₃₋₆ alkyl; and all other variables are as originally defined or as defined in any one of the preceding embodiments.

A seventh embodiment of the process of the invention is the process as originally described, wherein R³ is t-butyl; and all other variables are as originally defined or as defined in any one of the first five embodiments. As may be seen by reference to Examples 4-7 below, it has been found that the process of the invention provides higher yields and better regioselectivity when R³ is a branched —C₃₋₆ alkyl (e.g., t-butyl) compared to the case where R³ is not branched (e.g., methyl).

An eighth embodiment of the process of the invention is the process as originally described, wherein R⁴ is methyl; and all other variables are as originally defined or as defined in any one of the preceding embodiments.

A ninth embodiment of the process of the invention is the process as originally described, wherein each R^{a} set forth in the definitions of R¹ and R² is independently —H or —C₁₋₄ alkyl; and all other variables are as originally defined or as defined in any one of the preceding embodiments. In an aspect of this embodiment, each R^{a} is independently —H or —C₁₋₃ alkyl. In another aspect of this embodiment, each R^{a} is independently —H, methyl, or ethyl. In still another aspect of this embodiment, each R^{a} is independently —H or methyl.

It is understood that the definition of any one of R¹, R², R³, R⁴, R^{a}, R^{b}, P^{b}, R^{c}, P^{c}, HetA, HetB, and HetC as originally set forth or as defined in any of the foregoing embodiments of the process, or aspects thereof, can be combined with the definition of any one or more of the others of R¹, R², R³, R⁴, R^{a}, R^{b}, P^{b}, R^{c}, P^{c}, HetA, HetB, and HetC as originally set forth-or as defined in one of the foregoing embodiments or aspects thereof. Each such possible combination not expressly described above can be incorporated into the process of the invention as described above, and each represents an additional embodiment of the process of the present invention.

The amidine reactants of Formula II employed in the process of the invention can be prepared by direct condensation of nitrites with ammonia or aluminum amides, as described, for example in Granik, Russ. Chem. Rev. 1983, 52: 377, and in Gautier et al., In The Chemistry of Amidines and Imidates, edited by S. Patai, (Wiley, New York, 1975) pp. 283-348. Suitable amidines can also be prepared by conversion of nitrites to imidates, followed by conversion of the imidates to amidines with amines, as described, for example, in Roger et al., In The Chemistry of Imidates; edited by S. Patai, (Wiley, New York, 1960) p. 179. Amidines can also be prepared by conversion of nitrites to n-hydroxy amidines, followed by NO reduction, as described, for example, in Batt et al., J. Org. Chem. 2000, 65: 8100, and references cited therein. Amidines can also be prepared by activation of amides, followed by treatment with amines, as described, for example, in Charette et al., Tetrahedron Lett. 2000, 41: 1677, and references cited therein.

The amidines can optionally be employed in the form of acid salts, which can be formed by contacting at least an equivalent of an acid with the amidine and then recovering the acid salt. Either inorganic or organic acids can be used. Suitable inorganic acids include hydrochloric acid, hydrobromic acid, and sulfuric acid. Suitable organic acids include alkylcarboxylic acids, alkylsulfonic acids and arylsulfonic acids. Exemplary organic acids include acetic acid, trifluoroacetic acid, toluenesulfonic acid, benzenesulfonic acid, methanesulfonic acid, and trifluoromethanesulfonic acid.

The dioxyfumarate reactants of Formula III employed in the process of the invention can be prepared by the Claisen condensation of a dialkyl oxalate with an alkyl α-(alkyl- or aryl- or arylalkyloxy-)acetate: 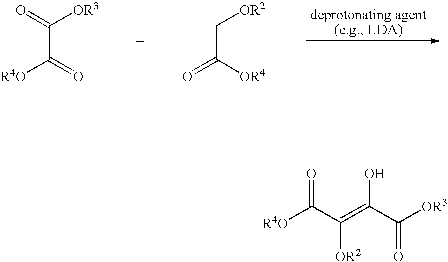
The Claisen condensation is further described in, for example, March, Advanced Organic Chemistry, 4th edition, (Wiley-Interscience, New York, 1992), pp. 491-493.

The organic solvent employed in the reaction step of the process of the invention can be any organic substance which under the reaction conditions employed is in the liquid phase, is chemically inert, and will dissolve, suspend, and/or disperse the reactants and the base so as to bring the reactants and base into contact and permit the reaction to proceed. Polar organic solvents are typically suitable for use in the reaction step of the process of the invention. In one embodiment, the solvent is selected from the group consisting of alcohols, ethers, and tertiary amides. Exemplary solvents include methanol, ethanol, propanol, isopropanol, n-butanol, isobutanol, THF, DME, MTBE, ethyl ether, n-butyl ether, dioxane, DMF, and DMAC.

A class of solvents suitable for use in the reaction step of the process of the invention is the solvents selected from the group consisting of C₁-C₆ alkyl alcohols, dialkyl ethers wherein each alkyl is independently a C₁-C₆ alkyl, C₄-C₈ cyclic ethers and diethers, C₆-C₈ aromatic ethers, and N,N-di-C₁-C₆ alkyl tertiary amides of C₁-C₆ alkylcarboxylic acids. A sub-class of suitable solvents consists the C₁-C₆ alkyl alcohols. When an alcohol is used as the solvent, methanol is particularly suitable when R⁴ is methyl, and ethanol is particularly suitable when R⁴ is ethyl.

The process of the invention requires the presence of a base. While not wishing to be bound by any particular theory, it is believed that the base deprotonates both the amidine II and the dioxyfumarate III, thereby increasing reactivity. When amidine II is employed in the form of an acid salt, the base also acts to neutralize the acid salt. Suitable bases include metal carbonates, metal bicarbonates, metal alkoxides, tertiary alkyl amines, tertiary cyclic amines, and diazabicycloalkenes. Exemplary bases include Na₂CO₃, K2CO₃, NaHCO₃, KHCO₃, TEA, NMM, NMP, LiOMe, LiOEt, LiOt-Bu, NaOMe, NaOEt, NaOt-Bu, KOMe, KOEt, KOt-Bu, Mg(OMe)₂, Mg(OEt)₂, DBU, DBN, and DABCO.

A class of bases suitable for use in the reaction step of the process of the invention is the bases selected from the group consisting of alkali metal C₁₋₄ alkoxides, tri-(C₁₋₄ alkyl)amines, tertiary cyclic amines, and diazabicycloalkenes. A sub-class of bases particularly suitable for use in the reaction step of the process of the invention is NaOMe, NaOEt, DBU, and TEA. When a metal alkoxide is employed, a methoxide is particularly suitable when R³ and R⁴ are both methyl, and an ethoxide is particularly suitable when R³ and R⁴ are both ethyl. More particularly, it is preferred to employ an alkali metal methoxide (e.g., NaOMe) as the base and methanol as the solvent when R³ and R⁴ are both methyl, and to employ an alkali metal ethoxide (e.g., NaOEt) and ethanol when R³ and R⁴ are both ethyl. The use of these combinations of base and solvent will avoid the possibility of transesterification (i.e., replacement of alkyl group R³ with a different alkyl group from R⁴ in the product) which can otherwise occur to some extent particularly when R³ is a linear alkyl group (v. a branched alkyl).

The dioxyfumarate of Formula III can be employed in the reaction step of the process of the invention in any proportion with respect to the amidine of Formula II which will result in the formation of at least some of Compound I, but it is typically employed in an amount that can optimize conversion of amidine II and formation of Compound I. For example, the dioxyfumarate m can suitably be employed in an amount of at least about 1 equivalent per equivalent of the amidine of Formula II. In one embodiment, the dioxyfumarate m is employed in an amount in a range of from about 1 to about 3 (e.g., from about 1 to about 2) equivalents per equivalent of amidine II. In another embodiment, the dioxyfumarate III is employed in an amount in a range of from about 1 to about 1.5 equivalents per equivalent of amidine II.

The base is suitably employed in the reaction step of the process of the invention in an amount of at least about one equivalent per equivalent of amidine II. In the event an acid salt of the amidine is employed in the process, an additional amount of base is needed to partially or completely neutralize the salt. Typically at least about one additional equivalent of base is employed to neutralize the salt. In one embodiment, the base is employed (i) in an amount of at least about 2 equivalents per equivalent of the amidine of Formula II when the free amidine is employed and (ii) in an amount of at least about 3 equivalents per equivalent of the amidine of Formula II when the amidine salt is employed. The amounts of base set forth in the preceding embodiment are typically necessary for complete conversion of the reactants.

The reaction between amidine II and dioxyfumarate III in the presence of base can be conducted at any temperature at which the reaction (condensation) forming Compound I can be detected. The reaction can suitably be conducted at a temperature in a range of from about −25 to about 100° C., and is typically conducted at a temperature in a range of from about −5 to about 50° C. In one embodiment, the temperature is in a range of from about 0 to about 40° C. (e.g., from about 15 to about 30° C.).

The reaction can be conducted by forming a mixture (typically a solution) of amidine II and dioxyfumarate III in the selected organic solvent at a temperature below the desired reaction temperature, charging the base thereto, and then bringing the resulting mixture to reaction temperature and maintaining the mixture at reaction temperature (optionally with agitation such as stirring) until the reaction is complete or the desired degree of conversion of the reactants is achieved. The order of addition of the reactants and solvent to the reaction vessel is typically not critical; i.e., they can be charged concurrently or sequentially in any order. For example, the amidine II can first be dissolved in the organic solvent, and the solution charged to the reaction vessel, followed by addition of dioxyfumarate III. The reaction time can vary widely depending upon, inter alia, the reaction temperature and the choice and relative amounts of reactants and base, but the reaction time for complete conversion is typically in a range of from about 6 to about 96 hours (e.g., from about 8 to about 48 hours). Compound I can subsequently be isolated (alternatively referred to as recovered) from the reaction mixture using conventional procedures, such as by aqueous acidification of the mixture to precipitate the product, followed by filtration, washing, and drying of the precipitate.

The present invention also includes a process which comprises the step of reacting an amidine of Formula II and a dioxyfumarate of Formula III to obtain a Compound of Formula I, as defined and described above, wherein R² is —C₁₋₆ alkylene-aryl, where the aryl is optionally substituted with from 1 to 5 substituents each of which is independently halo, —OH, —CN, —NO₂, —CO₂R^{a}, —C₁₋₆ alkyl, —O—C₁₋₆ alkyl, —C₁₋₆ alkylene-OH, or —C₁₋₆ alkylene-O—C₁₋₆ alkyl; and wherein the process further comprises treating the compound of Formula I with a hydroxy deprotecting agent to obtain a compound of Formula V-A: 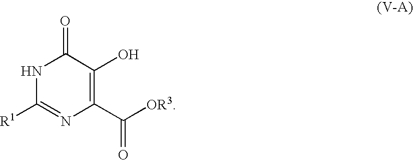
A suitable hydroxy deprotecting agent is hydrogen, wherein treating comprises contacting Compound I with hydrogen optionally in the presence of a hydrogenation catalyst. The hydrogenolysis of the R² (i.e., arylalkyl) group can suitably be accomplished with hydrogen and a Pd-containing catalyst (e.g., Pd/C). Acids can also be employed as hydroxy deprotecting agents. Suitable acids include the mineral acids, Lewis acids, and organic acids noted earlier in the discussion of the removal of amine protective groups. When an acid is employed, conditions are suitably selected and controlled to avoid or minimize concurrent hydrolysis of either the R¹ group or the —C(═O)OR³ ester group. The selection of suitable acids and acid treatment conditions can be done without undue experimentation by those of ordinary skill in the art. Further description of suitable acids and conditions for their use can be found in Greene and Wuts and in McOmie (both cited above). Embodiments of this process include the process as just described incorporating any one or more of the substituent definitions, reagents and process conditions set forth above in any of the embodiments, aspects or features of the reaction of amidine II with dioxyfumarate Emi.

In another embodiment of the process for obtaining compound V-A, in the event the R¹ group and/or the C(═O)OR³ group in the molecule is unstable or reactive under the conditions employed for removing R², the process further comprises protecting the R¹ group and/or the C(═O)OR³ group in Compound I prior to treating with a hydroxy deprotecting agent and then deprotecting the R¹ group and/or the C(═O)OR³ group subsequent to the removal of R².

The present invention also includes a process which comprises the step of reacting an amidine of Formula II and a dioxyfumarate of Formula III to obtain a Compound of Formula I, as defined and described above, wherein R² is —C₁₋₆ alkylene-aryl, where the aryl is optionally substituted with from 1 to 5 substituents each of which is independently halo, —OH, —CN, —NO₂, —CO₂R^{a}, —C₁₋₆ alkyl, —O—C₁₋₆ alkyl, —C₁₋₆ alkylene-OH, or —C₁₋₆ alkylene-O—C₁₋₆ alkyl; and wherein the process further comprises treating Compound I with a strong acid to obtain a compound of Formula V-B: 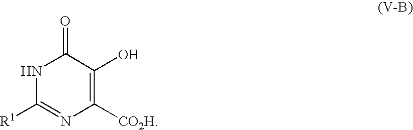
Suitable acids include concentrated inorganic acids such as HCl, HBr, sulfuric acid, trifluoroacetic acid, and phosphoric acid. Conditions are suitably selected and controlled to avoid or minimize concurrent hydrolysis of the R¹ group. The selection of suitable acids and acid treatment conditions can be done without undue experimentation by those of ordinary skill in the art. Embodiments of this process include the process as just described incorporating any one or more of the substituent definitions, reagents and process conditions set forth above in any of the embodiments, aspects or features of the reaction of amidine II with dioxyfumarate III.

In another embodiment of the process for obtaining compound V-B, in the event the R¹ group in the molecule is unstable or reactive under the conditions employed for hydrolysis of R² and the C(═O)OR³ ester, the process further comprises protecting the R¹ group in Compound I prior to treating with acid and then deprotecting the R¹ group and/or the C(═O)OR³ group subsequent to the acid treatment step.

The present invention also includes a process for preparing a compound of Formula VI: 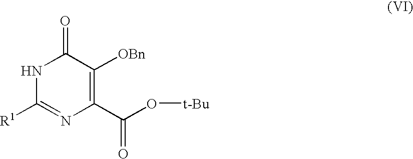
which comprises reacting an amidine of Formula II, or an acid salt thereof: 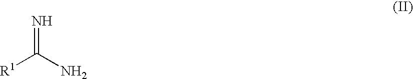
with a benzyloxyhydroxyfumarate of Formula Ia: 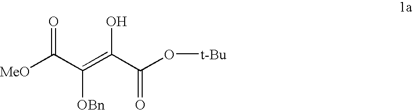
in an organic solvent and in the presence of a base to obtain the compound of Formula VI; wherein R¹ is as originally defined above.

Embodiments of the process for preparing Compound VI include the process as originally set forth incorporating any one or more of the following aspects (a) to (f):

(a-i) R¹ is as defined above in the first embodiment;
(a-ii) R¹ is as defined above in the second embodiment;
(a-iii) R¹ is as defined above in the third embodiment;
(b-i) the organic solvent is a polar organic solvent selected from the group consisting of alcohols, ethers, and tertiary amides;
(b-ii) the organic solvent is a C₁₋₆ alkyl alcohol;
(b-iii) the organic solvent is methanol (particularly suitable when employed in combination with an alkali metal methoxide, such as NaOMe, as base);
(c-i) the base is selected from the group consisting of metal carbonates, metal bicarbonates, metal alkoxides, tertiary alkyl amines, tertiary cyclic amines, and diazabicycloalkenes;
(c-ii) the base is selected from the group consisting of alkali metal C₁₋₄ alkoxides, tri-(C₁₋₄ alkyl)amines, tertiary cyclic amines, and diazabicycloalkenes;
(c-iii) the base is an alkali metal methoxide, such as NaOMe (particularly suitable when employed in combination with MeOH as solvent);
(c-iv) the base is NaOMe, DBU or TEA;
(d-i) the benzyloxyhydroxyfumarate 1a is employed in an amount of at least about 1 equivalent per equivalent of the amidine of Formula II;
(d-ii) the benzyloxyhydroxyfumarate 1a is employed in an amount in a range of from about 1 to about 3 (e.g., from about 1 to about 2) equivalents per equivalent of the amidine II;
(d-iii) the benzyloxyhydroxyfumarate 1a is employed in an amount in a range of from about 1 to about 1.5 equivalents per equivalent of amidine II;
(e-i) the base is employed (i) in an amount of at least about 2 equivalents per equivalent of the amidine of Formula II when the free amidine is employed and (ii) in an amount of at least about 3 equivalents per equivalent of the amidine of Formula II when the amidine salt is employed;
(f-i) the reaction is conducted at a temperature in a range of from about −25 to about 100° C.;
(f-ii) the reaction is conducted at a temperature in a range of from about −5 to about 50° C.;
(f-iii) the reaction is conducted at a temperature in a range of from about 0 to about 40° C. (e.g., from about 15 to about 30° C.).

A further embodiment is the process for preparing Compound VI as originally described, wherein, when R¹ in Compound VI is —C₁₋₆ alkylene-N(R^{b}R^{c}) in which (a) R^{b} is P^{b} and R^{c} is P^{c} or (b) R^{b} is P^{b} and R^{c} is other than P^{c}, the process optionally further comprises treating Compound VI with one or more anine deprotecting agents (concurrently or sequentially) to obtain a primary or a secondary amine derivative thereof. Aspects of this embodiment include the process as just described wherein the step of reacting amidine II and benzyloxyhydroxyfumarate 1a incorporates one or more of aspects (b) to (f).

Still another embodiment is the process for preparing Compound VI as originally described, which further comprises treating Compound VI with a hydroxy deprotecting agent to obtain a compound of Formula VII-A: 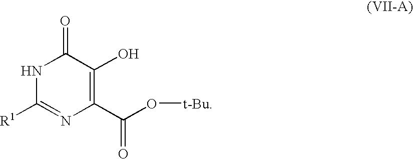
Suitable hydroxy deprotecting agents are the same as those employed to obtain Compound V-A by treating Compound I, as set forth above. Aspects of this embodiment include the process for obtaining Compound VII-A as just described, wherein the step of reacting amidine II and benzyloxyhydroxyfumarate 1a to obtain Compound VI incorporates one or more of aspects (a) to (f) set forth above. In another aspect of this embodiment, in the event the R¹ group in the molecule is unstable or reactive under the conditions employed for removing Bn, the process further comprises protecting the R¹ group in Compound VI prior to treating with a hydroxy deprotecting agent and then deprotecting the R¹ group subsequent to the removal of Bn.

Still another embodiment is the process for preparing Compound VI as originally described, which further comprises treating Compound VI with a strong acid to obtain a compound of Formula VII-B (equivalent to Compound V-B): 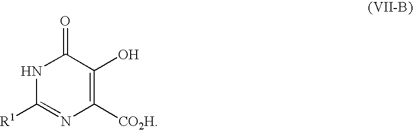
Suitable acids include concentrated inorganic acids such as HCl or sulfuric acid. Aspects of this embodiment include the process for obtaining Compound VII-B as just described, wherein the step of reacting amidine II and benzyloxyhydroxyfumarate 1a to obtain Compound VI incorporates one or more of aspects (a) to (f) set forth above. In another aspect of this embodiment, in the event the R¹ group in the molecule is unstable or reactive under the conditions employed for hydrolysis of Bn and the t-butyl ester, the process further comprises protecting the R¹ group in Compound VI prior to treating with acid and then deprotecting the R¹ group subsequent to the acid treatment step.

The present invention also includes a compound of Formula III-A: 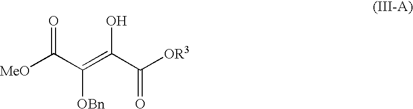
wherein R³ is —C₁₋₆ alkyl. An embodiment of the invention is a compound of Formula III-A, wherein R³ is a branched —C₃₋₆ alkyl. Another embodiment of the invention is a compound of Formula III-A, wherein R³ is t-butyl.

Still other embodiments of the present invention include any and all of the processes as originally defined and described above and any embodiments or aspects thereof as heretofore defined, further comprising isolating (which may be alternatively referred to as recovering) the compound of interest (i.e., compounds of Formula T. IV, V-A, V-B, VI, VII-A, and VII-B) from the reaction medium.

The progress of any reaction step set forth herein can be followed by monitoring the disappearance of a reactant (e.g., amidine II or dioxyfumarate III) and/or the appearance of the desired product (e.g., Compound I) using such analytical techniques as TLC, HPLC, IR, NMR or GC.

Compounds I, IV, V-A, V-B, VI, VIIA and VIIB are useful as intermediates in the preparation of pharmacologically active compounds. The compounds are, for example, useful as intermediates in the preparation of 5-hydroxy-6-oxo-1,6-dihydropyrimidine-4-carboxamides disclosed in WO 03/035077. The carboxamides in WO 03/035077 are HIV integrase inhibitors useful, inter alia, in treating HIV infection. In some cases, the carboxamides can be prepared directly from the compounds of Formula I, etc. by coupling (condensing) the compounds with suitable amines (e.g., benzylamines). In other cases, some pre- and/or post-coupling chemical modification is necessary (e.g., conversion of OR² to OH before or after the coupling of the amine with Compound I) to afford the desired integrase inhibitor. Example 14 below is representative of the use of Compounds I, IV, etc. as intermediates in the synthesis of these integrase inhibitors.

As used herein, the term “alkyl” refers to any linear or branched chain alkyl group having a number of carbon atoms in the specified range. Thus, for example, “C₁₋₆ alkyl” (or “C₁-C₆ alkyl”) refers to all of the hexyl alkyl and pentyl alkyl isomers as well as n-, iso-, sec- and t-butyl, n- and isopropyl, ethyl and methyl. As another example, “C₁₋₄ alkyl” refers to n-, iso-, sec- and t-butyl, n- and isopropyl, ethyl and methyl.

The term “-alkylene-” refers to any linear or branched chain alkylene (or alternatively “alkanediyl”) having a number of carbon atoms in the specified range. Thus, for example, “—C₁₋₆ alkylene-” refers to the C₁ to C₆ linear or branched alkylenes. A class of alkylenes of particular interest with respect to the invention is —(CH₂)₁₋₆-, and sub-classes of particular interest include —(CH₂)₁₋₄-, —(CH₂)₁₋₃-, —(CH₂)₁₋₂-, and —CH₂—. Also of interest is the alkylene —CH(CH₃)—.

The term “halogen” (or “halo”) refers to fluorine, chlorine, bromine and iodine (alternatively referred to as fluoro, chloro, bromo, and iodo).

Unless expressly stated to the contrary, an “unsaturated” ring is a partially or fully unsaturated ring.

Unless expressly stated to the contrary, all ranges cited herein (i.e., process ranges such as a temperature range and ranges defined in the compounds set forth herein) are inclusive; i.e., the range includes the values for the upper and lower limits of the range as well as all values in between. Thus, for example, a heterocyclic ring described as containing from “1 to 4 heteroatoms” means the ring can contain 1, 2, 3 or 4 heteroatoms. It is also to be understood that any range (e.g., a temperature range) cited herein includes within its scope all of the sub-ranges within that range. Thus, for example, a heterocyclic ring described as containing from “1 to 4 heteroatoms” is intended to include as aspects thereof, heterocyclic rings containing 2 to 4 heteroatoms, 3 or 4 heteroatoms, 1 to 3 heteroatoms, 2 or 3 heteroatoms, 1 or 2 heteroatoms, 1 heteroatom, 2 heteroatoms, and so forth.

When any variable (e.g., R^{a}) occurs more than one time in any constituent or in Formula I or Formula II or in any other formula depicting and describing compounds employed or included in the invention, its definition on each occurrence is independent of its definition at every other occurrence. Also, combinations of substituents and/or variables are permissible only if such combinations result in stable compounds.

Unless clear from the context to the contrary, a reference herein to “equivalent” or “equivalents” means molar equivalent(s).

The term “substituted” (e.g., as in “the aryl is optionally substituted with from 1 to 5 substituents . . . ”) includes mono- and poly-substitution by a named substituent to the extent such single and multiple substitution (including multiple substitution at the same site) is chemically allowed. Unless expressly stated to the contrary, substitution by a named substituent is permitted on any atom in a ring (e.g., aryl, a heteroaromatic ring, or a saturated heterocyclic ring) provided such ring substitution is chemically allowed and results in a stable compound.

Any heterocyclic ring substituent defined herein can be attached to the rest of the compound via either a ring carbon atom or a ring heteroatom, provided such attachment is chemically allowed and results in a stable compound.

As would be recognized by one of ordinary skill in the art, depending upon such factors as the reaction temperature, the choice and relative amounts of the reactants, and the choice of solvent and base, the compound of Formula III might exist entirely or partly in a tautomeric form in the process of the present invention, including a tautomer of Formula III-A or III-B: 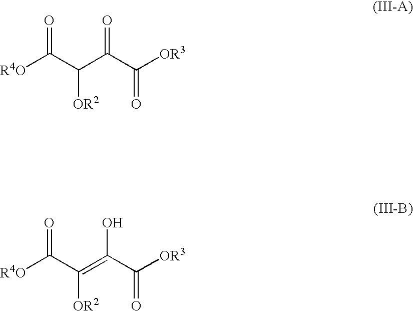
It is understood that, for the purposes of the present invention, a reference herein to a compound of Formula III is a reference to compound III per se, or to any one of its tautomers per se (e.g., III-A or III-B), or to mixtures of two or more thereof.

As would also be recognized by one of ordinary skill in the art, the compound of Formula I might also exist and be isolatable in a tautomeric form, including a tautomer of Formula I-A: 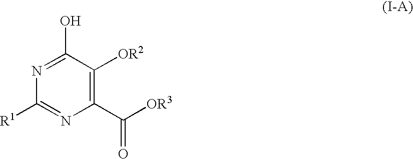
It is understood that, for the purposes of the present invention, a reference herein to a compound of Formula I is a reference to compound I per se, or to any one of its tautomers per se (e.g., I-A), or to mixtures thereof.

Abbreviations used in the instant specification include the following:
Ac=acetyl
Alloc=allyloxycarbonyl
Bn=benzyl
BOC or Boc=t-butyloxycarbonyl
t-Bu=tertiary butyl
Cbz=carbobenzoxy (alternatively, benzyloxycarbonyl)
DABCO=1,4-diazabicyclo[2.2.2]octene
DBN=diazabicyclononene
DBU=1,8-diazabicyclo[5.4.0]undec-7-ene
DMAC=N,N-dimethylacetamide
DME=1,2-dimethoxyethane
DMF=N,N-dimethylformamide
EDC=1-ethyl-3-(3-dimethylaminopropyl) carbodiimide
Et=ethyl
EtOAc=ethyl acetate
HOBt=1-hydroxy benzotriazole hydrate
HPLC=high performance liquid chromatography
LCMS=liquid chromatography mass spectroscopy
LDA=lithium diisopropylamide
Me=methyl
MeOH=methanol
MTBE=methyl tert-butyl ether
NMM=N-methylmorpholine
NMP=N-methylpyrrolidinone
NMR=nuclear magnetic resonance
Ph=phenyl
Pr=propyl
i-Pr=isopropyl
rt=room temperature
TEA=triethylamine
THF=tetrahydrofuran
TLC=thin layer chromatography
UV=ultraviolet

The following examples serve only to illustrate the invention and its practice. The examples are not to be construed as limitations, on the scope or spirit of the invention.

### EXAMPLE 1

### Preparation of 2-benzyloxy-3-hydroxy Fumarate 4-tert-butyl Ester 1-methyl Ester (1a)

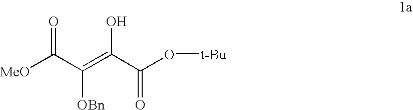

LDA was generated by addition of n-BuLi (60 mL, 2.5 M in hexanes, 150 mmol) to diisopropylamine (21.0 mL, 150 mmol) in THF (60 mL) at 0° C., and aged for 10 minutes. In a separate flask, a mixture of methyl tert-butyl oxalate (24.0 g, 150 mmol) and methyl α-benzyloxy acetate (18.0 g, 100 mmol) in THF (240 mL) was cooled to −78° C. The LDA was then added quickly by cannula, and the resulting mixture stirred for 1 hour at −78° C., then warmed to room temperature over 1 hour. The mixture was then quenched with cold aqueous HCl (200 mil, 1 M), and the reaction was diluted with EtOAc (200 mL). The layers were separated, and the aqueous was back-washed with EtOAc (2×100 mL). The resulting organics were dried (MgSO₄), filtered and stripped, then purified by column chromatography on silica-gel (eluants 1%-60% EtOAc/hexanes) to give 22.2 g (72%) of clean 1a. ¹H and ¹³C NMR were complex due to multiple enol tautomers and enol olefin configurations. The purified product provided a single spot by TLC analysis. Fumarate 1a is typically used in subsequent reactions immediately after purification, due to decomposition.

### EXAMPLE 2

### Preparation of 5-benzyloxy-6-oxo-2-cyclopropyl-1,6-dihydropyrimidine-4-carboxylic Acid tert-butyl Ester

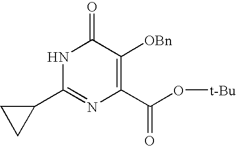

NaOMe (6.8 mL, 25 wt % solution in MeOH, 30 mmol) was added to a solution of cyclopropane-1-caboximidamide HCl (1.21 g, 10.0 mmol) and fumarate reagent 1a (4.60 g, 15.0 mmol) in MeOH (16.6 mL) at 0° C. The mixture was warmed to room temperature, then stirred for 30 hours. After dilution with MeOH (5 mL) and cooling to 0° C., 1N HCl (40 mL) was added and the product was precipitated from the mixture. The solid was washed with 10 mL of cold 9:1H₂OMeOH and dried giving 3.14 g of the title product (92% isolated yield) of >98% pure (HPLC area percent purity at 210 nm) as a white crystalline solid with a melting point of 164.0-164.5° C.

¹H-NMR (400 MHz, CDCl₃) d 12.98 (1H, br s), 7.46-7.49 (2H, m), 7.30-7.38 (m, 3h), 5.24 (2H, s), 1.89-1.95 (1H, m), 1.52 (9H, s), 1.24-1.29 (2H, M), 1.05-1.10 (2H, m); ¹³C NMR (100 MHz, DMSO-d₆) 164.3, 159.7, 159.5, 145.7, 139.4, 137.3, 128.6, 128.3 (2 peaks), 82.6, 73.4, 27.9, 13.5, 10.0 ppm.

### EXAMPLES 3-13

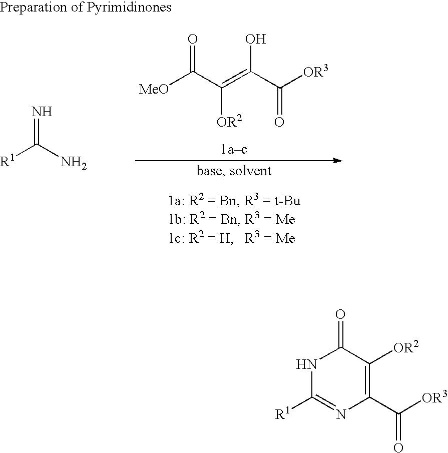

A series of pyrimidinones was prepared using procedures similar to the procedure set forth in Example 2.

The reactants, reaction conditions, yields, and physical characterization data for these preparations are provided in the following Table:

| Example | | | | | |
|---|---|---|---|---|---|
| No. | Fumarate | R¹ | Conditions | Yield² | Physical Data |
| 3 | 1c | Ph | Three runs: | Trace | |
| | | | NaOMe, rt, 16 hrs | | |
| | | | Et₃N, 60 C., 16 hrs | | |
| | | | DBU, 60 C., 8 hrs | | |
| 4 | 1b | Ph | DBU, 60 C., 8 hrs | 65 | ¹H-NMR(400MHz, CDCl₃) d |
| | | | | | 12.71(1H, br s), 8.19(2H, m), |
| | | | | | 7.40-7.55(5H, m), 7.34-7.40(m, |
| | | | | | 3h), 5.38(2H, s), 3.93(3H, s); |
| | | | | | ¹³C NMR(100MHz, DMSO-d6) |
| | | | | | 165.1, 160.1, 152.7, 143.5, |
| | | | | | 142.1, 137.0, 131.9, 129.0, |
| | | | | | 128.7(2 peaks), 128.6, 128.1, |
| | | | | | 73.9, 52.8ppm. |
| 5 | 1a | Ph | DBU, 60 C., 8 hrs | na (92) | ¹H-NMR(400MHz, CDCl₃) d |
| | | | | | 12.32(1H, br s), 8.16(2H, m), |
| | | | | | 7.33-7.53(m, 8h), 5.36(2H, s), |
| | | | | | 1.57(9H, s); ¹³C NMR(100MHz, |
| | | | | | CDCl₃-d6) 163.8, 162.0, |
| | | | | | 151.8, 146.6, 141.0, 136.7, |
| | | | | | 131.6, 131.3, 128.8, 128.4, |
| | | | | | 128.2(2 peaks), 127.6, 83.4, |
| | | | | | 74.0, 28.0ppm. |
| 6 | 1a | Ph | Et₃N, 60 C., 24 hrs | na (82) | |
| 7 | 1a | Ph | NaOMe, rt, 24 hrs | 84 (89) | |
| 8 | 1a | Me | same as Ex. 7 | 84 | ¹H-NMR(400MHz, CDCl₃) d |
| | | | | | 7.47(2H, m), 7.33-7.39(3H, m), |
| | | | | | 5.26(2H, s), 2.49(3H, s), |
| | | | | | 1.53(9H, s); ¹³C NMR(100MHz, |
| | | | | | DMSO-d6) 164.0, 161.4, 159.7, |
| | | | | | 155.5, 144.6, 140.2, 137.3, |
| | | | | | 128.7, 128.5, 83.1, 73.6, 28.0, |
| | | | | | 20.9ppm. |
| 9 | 1a | H | same as Ex. 7 | 93 | ¹H-NMR(400MHz, CDCl₃) d |
| | | | | | 8.40(1H, br s), 7.45(2H, m), |
| | | | | | 7.31-7.39(3H, m), 5.36(2H, s), |
| | | | | | 1.53(9H, s); ¹³C NMR(100MHz, |
| | | | | | DMSO-d6) 164.0, 159.1, |
| | | | | | 145.6, 145.1, 142.5, 137.2, |
| | | | | | 128.7, 128.6, 128.5, 83.0, 73.6, |
| | | | | | 28.1ppm. |
| 10 | 1a | 4-pyridyl | NaOMe, rt, 30 hrs | 96 | ¹H-NMR(400MHz, CDCl₃) |
| | | | | | 8.71(2H, d, m), 8.13(2H, d, m), |
| | | | | | 7.49, (m, 2h), 7.383-7.44(m, |
| | | | | | 3H), 5.39(2H, s), 1.58(9H, s); |
| | | | | | ¹³C NMR(100MHz, CDCl₃-d6) |
| | | | | | 163.3, 162.1, 150.6, 149.3, |
| | | | | | 146.4, 142.4, 138.4, 136.2, |
| | | | | | 128.5, (2 peaks), 128.0, 121.0, |
| | | | | | 83.8, 74.3, 28.0ppm. |
| 11 | 1a | 3-NO₂Ph | NaOMe, rt, 48 hrs | 88 | ¹H-NMR(400MHz, CDCl₃) d |
| | | | | | 9.90(1H, m), 8.58(1H, m), |
| | | | | | 8.34(1H, m), 7.55(1H, m), |
| | | | | | 7.42-7.45(2H, m), 7.35-7.40(3H, |
| | | | | | m), 5.38(2H, s), 1.57(9H, s); |
| | | | | | ¹³C NMR(100MHz, DMSO-d6) |
| | | | | | 163.9, 160.4, 151.1, 148.3, 141.2, |
| | | | | | 137.0, 134.2, 133.9, 130.7, |
| | | | | | 128.7(2 peaks), 128.5, 128.4, |
| | | | | | 126.1, 122.8, 83.2, 73.7, 28.0ppm. |
| 12 | 1a | 4-MeOPh | NaOMe (2 eq.), | 94 | ¹H-NMR(400MHz, CDCl₃) d |
| | | | rt, 30 hrs | | 12.73(1H, br s), 8.19(2H, m), |
| | | | | | 7.52(d, 2H, m), 7.33-7.41(m, |
| | | | | | 3H), 6.89(d, 2H, m), 5.33(2H, |
| | | | | | s), 3.79(s, 3H), 1.57(9H, s); ¹³C |
| | | | | | NMR(100MHz, CDCl₃-d6) |
| | | | | | 164.1, 162.6, 162.3, 151.9, |
| | | | | | 147.1, 140.5, 137.1, 129.6, |
| | | | | | 128.6, 128.5, 128.3, 123.9, |
| | | | | | 114.3, 83.4, 74.1, 55.5, 28.2ppm. |
| 13 | 1a | MeO | NaOMe, rt, 36 hrs | 50 | ¹H-NMR(400MHz, CDCl₃) d |
| | | | | | 11.10(1H, br s), 7.49(2H, m), |
| | | | | | 7.30-7.38(m, 3h), 5.17(2H, s), |
| | | | | | 4.00(3H, s), 1.52(9H, s); ¹³C |
| | | | | | NMR(100MHz, CDCl₃-d6) |
| | | | | | 163.7, 162.2, 152.8, 145.7, |
| | | | | | 137.7, 136.8, 128.2(2 peaks), |
| | | | | | 128.0, 83.3, 74.2, 55.5, 27.9ppm. |

Notes
¹The HCl salt of the amidine was employed in Examples 3-11; amidine free base was used in Example 12; and the sulfate salt(i.e., ½ H₂SO₄) was used in Example 13.
²HPLC assay yield is in parentheses. na denotes that the compound was not isolated under these conditions.

In Example 1, dimethoxydihydroxy fumarate 1c was reacted with benzamidine HCl in MeOH at room temperature in three separate runs using NaOMe, DBU, and Et₃N respectively as the base. A trace amount of the desired methyl 5-benzyloxy-6-oxo-2-phenyl-1,6-dihydropyrimidine-4-carboxylate 2 was formed (>10% by LCMS) with complete consumption of the starting materials. Amidine-incorporated products with lower than expected mass were obtained as the major products (by LCMS). In contrast, in Example 4, fumarate reagent 1b (1.5 eq.) was combined with benzamidine HCl and DBU (3 eq.) in MeOH at 60° C. for 8 hours to afford a 65% yield of the desired product 2 with a five-membered imidazole 3 as a major side product: 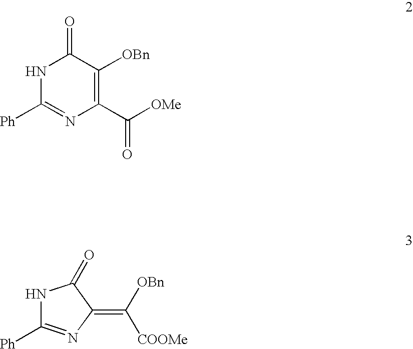
Futhermore, referring to Examples 5-7, substantially higher assay yields were obtained when using 1a. More particularly, in Example 7, 1a was reacted cleanly with benzamidine HCl using NaOMe (3 eq.) in MeOH at room temperature to give product 2 in 89% HPLC assay yield and 84% isolated yield after crystallization. Only a trace of the 5-membered ring 3 was present as the only side product.

### EXAMPLE 14

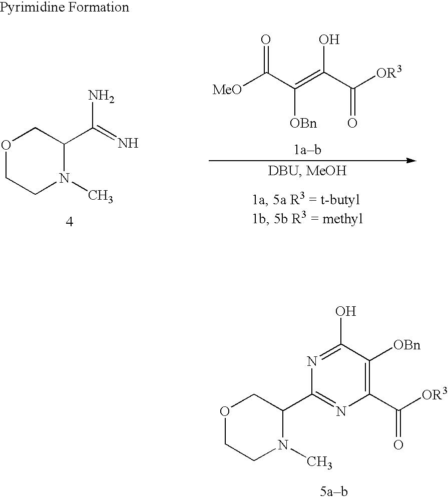 Part A—Preparation of Amidine 4
Step 1: Morpholine-3-nitrile

Morpholine (174 mL, 2 mol) in 325 mL ether was cooled over a −20° C. bath and tBuOCl (1 eq) was added over 30 min (internal temperature mostly <0° C.). After ageing for 30 minutes, NaOMe/methanol (1 eq) was added over 25 minutes. The mixture was allowed to warm slowly. At 35-40° C. there was a large exotherm with vigorous refluxing. The mixture was then aged for 1 hour at 40-43° C., allowed to cool to room temperature, and the slowly filtered, with rinsing of the solids with 1:1 Et₂O/MeOH. The orange-brown filtrate was rotary evaporated. The liquid residue was dissolved in 350 mL water (pH=12) and was cooled over an ice bath. KCN (1 eq) was added, then 260 mL conc HCl to give pH=4. After ageing for 2 hours at room temperature, the solution was adjusted to pH of 9 with 108 mL 22% NaOH and extracted with 3×250 mL dichloromethane. The organic layer was dried (Na₂SO₄) and concentrated to afford 110.9 g crude morpholine nitrile (50% crude yield). The aqueous layer was rotary evaporated, dried overnight under vacuum, and stirred with 500 mL dichloromethane for 1 hour. Filtration and evaporation afforded 47.1 g more product (21%). The solids were stirred again with 500 mL dichloromethane and filtered to afford 14.2 g more product (6%). The combined crude yield was 172 g (77%).

Step 2: 3-Cyano-4-methylmorpholine via reductive methylation

Morpholine nitrile (134.54 g, 1.2 Mol) from Step 1 was added to 3 L of dichloromethane, followed by addition of acetic acid (90 mL, 1.3 eq) at 0 C, and then HCHO (190 mL, 2.1 eq). Then NaCNBH₃ (165.8 g, 2.2 eq) was added over 20 minutes, followed by ageing for 40 minutes, and then addition of more NaCNBH₃ (5.0 g). NaOH (5N, 500 mL) was then added, the organic layer was separated, and the aqueous layer was washed with CH₂Cl₂ (300 mL). The organics were combined, dried with Na₂SO₄, and then passed through silica-gel (480 g), eluting with EtOAc (2.5 L). The organics were concentrated, ethanol (600 mL) was added, then HCl (600 mL, 2.0 M in Et₂O), followed by concentration. The concentrate was dissolved in EtOH (300 mL), then EtOAc (300 mL) was added slowly. The product crystallized as the HCl salt, which was filtered and washed with EtOAc (100 mL) to give 113.0 g isolated product (58% yield).

Step 3: N-Hydroxyamidine Formation

HCl salt product from Step 2 (105.7 g, 650 mmol) was suspended in EtOH (1050 mL), NH₂OH (160 mL, 50% aqueous) was added, and the mixture was warmed to circa 80 C for 16 hours. The mixture was then concentrated, followed by addtion of water (300 mL), NaHCO₃ (40 g) and NaCl (40 g). This mixture was extracted with EtOAc (3×300 mL), and then with CH₂Cl₂ (3×300 mL). The combined organics were dried and stripped, and then crystallized from EtOH (300 mL) and EtOAc (300 mL), giving 52.4 g (51% yield) of the n-hydroxyamidine.

Step 4: Amidine 4

n-Hydroxyamidine (49.0 g, 308 mmol) from Step 3 was dissolved in AcOH (980 ml) and Ac₂O (44 mL), then treated with Pd/C (7.35 g), hydrogenated at 20 psi, 20 C, and then filtered and concentrated. The resulting HOAc salt was recrystallized from CH₃CN to give 38.2 g (61% yield) of the title product.

Part B—Formation of Pyrimidines 5a-b

Run 1: Formation with dihydroxy fumarate 1a (R³=t-butyl)

The acetic acid salt of amidine 4 (0.125 g, 0.62 mmol) was mixed with fumarate reagent 1a (0.34 g, 0.93 mmol, 1.5 equiv), and this mixture was dissolved in MeOH (1.9 mL), and then treated with DBU (0.28 mL, 1.87 mmol, 3.0 equiv). After stirring overnight at room temperature, the assay yield of pyrimidine 5a was determined to be 93% by HPLC analysis.

Run 2: Formation with dihydroxy fumarate 1b (R³=methyl)

The reaction was conducted in the same manner as in Run 1, except that fumarate 1b was substituted for fumarate 1a. The yield of 5b was 65% versus the 93% assay yield of 5a.

### EXAMPLE 15

### Preparation of N-(4-fluorobenzyl)-5-hydroxy-1-methyl-2-(4-methylmorpho-lin-3-yl)-6-oxo-1,6-dihydropyrimidine-4-carboxamide (8) Step 1 Methylation

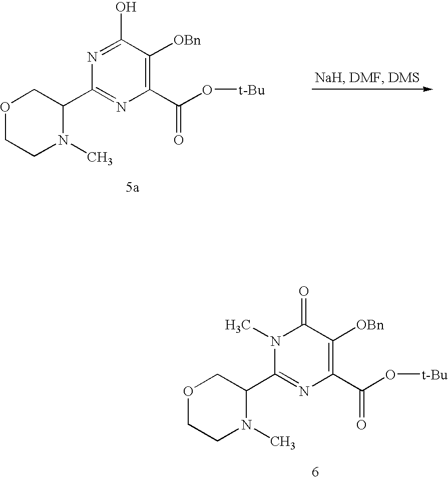

Pyrimidine 5a from Example 14 (0.70 g, 1.75 mmol) was dissolved in DMF (3.5 mL), then added to NaH (91 mg, 2.28 mmol) at 0 C. This mixture was then warmed to room temperature and stirred for 30 minutes. After cooling to 0 C, dimethyl sulfate (0.17 mL, 1.75 mmol) was added, and the mixture stirred for three hours. Then NH₄OH (1.0 mL) was added and the mixture was diluted with EtOAc (20 mL) and water (10 mL). After shaking, the organic portion was separated and the aqueous portion was washed with EtOAc (2×20 mL). The organic portion was dried (MgSO₄) and stripped. The product was then purified by column chromatography on silica-gel (gradient elution MTBE to 40% EtOAc/MTBE) to give 0.45 g of pure 6.
Step 2: Deprotection

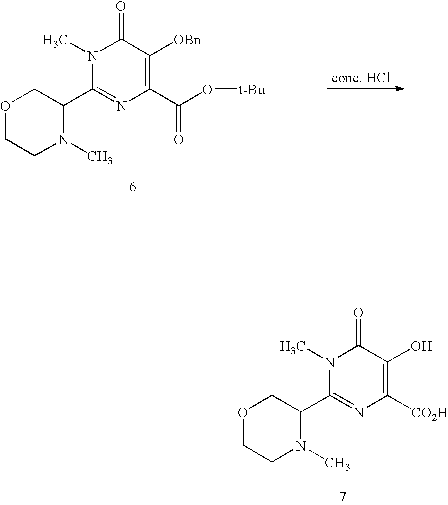

To pyrimidinone 6 (0.60 g, 1.44 mmol) in MeOH (6.0 mL), was added concentrated HCl (10.0 mL) at 0 C. This mixture was warmed to room temperature and stirred for 4 hours. The MeOH was removed by rotary evaporation, then more concentrated HCl (5.0 mL) was added and this was stirred for 2 hours. The solvent was then stripped on Hivac (16 mm Hg) at 60 C. The resulting solid was dissolved in MeOH (10 mL) at 50 C, then Et₂O was added (10 mL) and a white solid formed. After cooling and filtration, methylated pyrimidinone 7 was obtained (0.44 g, 100% yield, 90 A %) in approximately 90% yield.

Step 3: Amidation

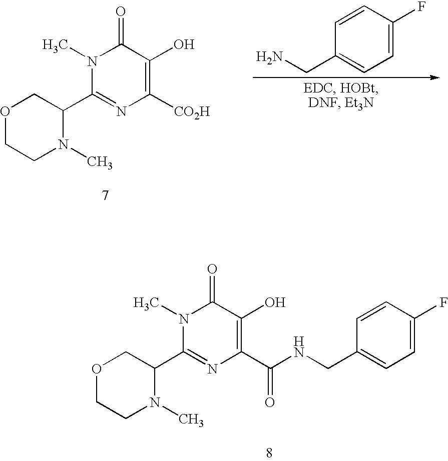

Pyrimidinone 7 (0.090 g, 0.30 mmol) was mixed with HOBt (cat.) and slurried in DMF (1.0 mL). 4-Fluorobenzylamine (0.14 mL, 1.2 mmol) and Et₃N (0.90 mL) were then added, and the resulting mixture was cooled to 0 C, followed by addition of EDC (0.12 g, 0.62 mmol). This mixture was warmed to room temperature and then stirred overnight to give a mixture containing title product 8. LC analysis showed a product of identical retention time to the product disclosed in Example 6 of WO 03/035077. The UV spectrum was also identical, and the LCMS exhibited the desired mass. The product was not isolated.

While the foregoing specification teaches the principles of the present invention, with examples provided for the purpose of illustration, the practice of the invention encompasses all of the usual variations, adaptations and/or modifications that come within the scope of the following claims.

1. A process for preparing a compound of Formula I: 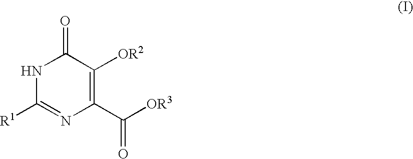
which comprises reacting an amidine of Formula II, or an acid salt thereof: 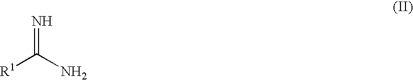
with a dioxyfumarate of Formula III: 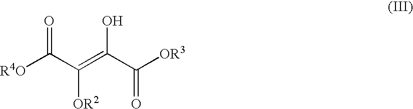
in an organic solvent and in the presence of a base to obtain the compound of Formula I;
wherein:
R¹ is:
(1) —H,
(2) —O—C₁₋₆ alkyl,
(3) —CO₂R^{a},
(4) —C₁₋₆ alkyl,
(5)-C₁₋₆ alkylene-CO₂R^{a},
(6)-C₁₋₆ alkylene-N(R^{b}R^{c}),
(7) —C₃₋₈ cycloalkyl, which is optionally substituted with from 1 to 4 substituents each of which is independently —OH, —CN, or —C₁₋₆ alkyl,
(8) —C₁₋₆ alkylene-C₃₋₈ cycloalkyl, where the cycloalkyl is optionally substituted with from 1 to 4 substituents each of which is is independently —OH, —CN, or —C₁₋₆ alkyl,
(9) aryl, which is optionally substituted with from 1 to 5 substituents each of which is independently halo, —OH, —CN, —NO₂, —CO₂R^{a}, —C₁₋₆ alkyl, —O—C₁₋₆ alkyl, —C₁₋₆ alkylene-OH, or —C₁₋₆ alkylene-O—C₁₋₆ alkyl,
(10) —C₁₋₆ alkylene-aryl, where the aryl is optionally substituted with from 1 to substituents each of which is independently halo, —OH, —CN, —NO₂, —CO₂R^{a}, —C₁₋₆ alkyl, —O—C₁₋₆ alkyl, —C₁₋₆ alkylene-OH, or —C₁₋₆ alkylene-O—C₁₋₆ alkyl,
(11) heterocycle, which is (i) optionally substituted with from 1 to 4 substituents each of which is independently halo or a —C₁₋₆ alkyl, and (ii) optionally mono-substituted with aryl or —C₁₋₆ alkylene-aryl, HetC or —C₁₋₆ alkylene-HetC, or
(12) —C₁₋₆ alkylene-heterocycle, where the heterocycle is (i) optionally substituted with from 1 to 4 substituents each of which is independently halo or a —C₁₋₆ alkyl, and (ii) mono-substituted with aryl or —C₁₋₆ alkylene-aryl, HetC or —C₁₋₆ alkylene-HetC;
R² is:
(1) —C₁₋₆ alkyl,
(2) aryl, which is optionally substituted with from 1 to 5 substituents each of which is independently halo, —OH, —CN, —NO₂, —CO₂R^{a}, —C₁₋₆ alkyl, —O—C₁₋₆ alkyl, —C₁₋₆ alkylene-OH, or —C₁₋₆ alkylene-O—C₁₋₁₆ alkyl, or
(3) —C₁₋₆ alkylene-aryl, where the aryl is optionally substituted with from 1 to 5 substituents each of which is independently halo, —OH, —CN, —NO₂, —CO₂R^{a}, —C₁₋₆ alkyl, —O—C₁₋₆ alkyl, —C₁₋₆ alkylene-OH, or —C₁₋₆ alkylene-O—C₁₋₆ alkyl;
R³ is —C₁₋₆ alkyl;
R⁴ is methyl or ethyl;
each R^{a} is independently —H or —C₁₋₆ alkyl;
R^{b} is:
(1) —C₁₋₆ alkyl,
(2) aryl, which is optionally substituted with from 1 to 4 substituents each of which is independently halo, —OH, —CN, —NO₂, —C₁₋₆ alkyl, or —O—C₁₋₆ alkyl, or
(3) P^{b}, which is an amine protective group;
R^{c} is:
(1) —C₁₋₆ alkyl,
(2) aryl, which is optionally substituted with from 1 to 4 substituents each of which is independently halo, —OH, —CN, —NO₂, —C₁₋₆ alkyl, or —O—C₁₋₆ alkyl, or
(3) P^{c}, which is an amine protective group;
each aryl is independently phenyl or naphthyl;
heterocycle is (i) a 4- to 8-membered, saturated or unsaturated monocyclic ring, (ii) a 7- to 12-membered bicyclic ring system, or (iii) an 11 to 16-membered tricyclic ring system; wherein each ring in (ii) or (iii) is independent of, bridged with, or fused to the other ring or rings and each ring is saturated or unsaturated, and the monocyclic ring, bicyclic ring system, or tricyclic ring system contains at least one carbon atom and from 1 to 6 heteroatoms independently selected from N, O and S; wherein any one or more of the nitrogen and sulfur heteroatoms is optionally oxidized; and
HetC is (i) a 5- or 6-membered heteroaromatic ring containing from 1 to 4 heteroatoms independently selected from N, O and S or (ii) a 4- to 7-membered saturated heterocyclic ring containing at least one carbon atom and from 1 to 4 heteroatoms independently selected from N, O and S; wherein any one or more of the nitrogen and sulfur heteroatoms in (ii) is optionally oxidized.

2. The process according to claim 1, wherein R¹ is:
(1) —H.
(2)—O—C₁₋₄ alkyl,
(3) —CO₂R^{a},
(4) —C₁₋₄ alkyl,
(5) —C₁₋₄ alkylene-CO₂R^{a},
(6) —C₁₋₄ alkylene-N(R^{b}R^{c}),
(7) —C₃₋₆ cycloalkyl, which is optionally substituted with from 1 to 3 substituents each of which is independently a —C₁₋₄ alkyl,
(8) —C₁₋₄ alkylene-C₃₋₆ cycloalkyl, where the cycloalkyl is optionally substituted with from 1 to 3 substituents each of which is independently a —C₁₋₄ alkyl,
(9)-phenyl, which is optionally substituted with from 1 to 3 substituents each of which is independently halo, —CN, —NO₂, —CO₂R^{a}, —C₁₋₄ alkyl, or —O—C₁₋₄ alkyl,
(10) —C₁₋₄ alkylene-phenyl, where the phenyl is optionally substituted with from 1 to 3 substituents each of which is independently halo, —CN, —NO₂, —CO₂R^{a}, —C₁₋₄ alkyl, or —O—C₁₋₄ alkyl,
(11)-HetA,
(12) —C₁₋₄ alkylene-HetA,
(13)-HetB, or
(14) —C₁₋₁₄ alkylene-HetB;
each R^{a} is independently —H or —C₁₋₄ alkyl;
R^{b} is:
(1) —C₁-4 alkyl,
(2) phenyl, which is optionally substituted with from 1 to 3 substituents each of which is independently halo, —NO₂, —C₁₋₁₄ alkyl, or —O—C₁₋₄ alkyl, or
(3) P^{b} which is an amine protective group selected from the group consisting of:
(i) —CH₂-phenyl, where the phenyl is optionally substituted with from 1 to 3 substituents each of which is independently halo, —NO₂, —C₁₋₄ alkyl, or —O—C₁₋₄ alkyl,
(ii)-C(═O)—C₁₋₄ alkyl,
(iii)-C(═O)—CH₂-phenyl,
(iv)-C(═O)—O—C₁₋₄ alkyl,
(v) —C(═O)—O—CH₂—CH═CH₂, or
(vi)-C(═O)—O—CH₂-phenyl, where the phenyl is optionally substituted with from 1 to 3 substituents each of which is independently halo, —NO₂, —C₁₋₄ alkyl, or —O—C₁₋₄ alkyl;
R^{c} is:
(1) —C₁₋₄ alkyl,
(2) phenyl, which is optionally substituted with from 1 to 3 substituents each of which is independently halo, —NO₂, —C₁₋₄ alkyl, or —O—C₁₋₄ alkyl, or
(3) P^{c} which is an amine protective group that independently has the same definition as P^{b} in the definition of R^{b} above;
HetA is a 5- or 6-membered heteroaromatic ring containing from 1 to 4 heteroatoms independently selected from N, O and S; wherein the heteroaromatic ring is (i) optionally substituted with from 1 to 3 substituents each of which is independently halo or a —C₁₋₄ alkyl and (ii) optionally mono-substituted with phenyl or —(CH₂)₁₋₂-phenyl;
HetB is a 4- to 7-membered saturated heterocyclic ring containing at least one carbon atom and a total of from 1 to 4 heteroatoms independently selected from N, O and S; wherein any ring sulfur, if present, is optionally substituted with oxo or dioxo; and wherein the heterocyclic ring is (i) optionally substituted with from 1 to 3 substituents each of which is independently halo or a —C₁₋₄ alkyl and (ii) optionally mono-substituted with phenyl, —(CH₂)₁₋₂-phenyl, HetC, or —(CH₂)₁₋₂-phenyl-HetC; and
HetC is (i) a 5- or 6-membered heteroaromatic ring containing from 1 to 4 heteroatoms independently selected from N, O and S or (ii) a 4- to 7-membered saturated heterocyclic ring containing at least one carbon atom and from 1 to 4 heteroatoms independently selected from N, O and S; wherein any one or more of the nitrogen and sulfur heteroatoms in (ii) is optionally oxidized.

3. The process according to claim 2, wherein R¹ is:
(1) —H,
(2) —C₁₋₄ alkyl,
(3) cyclopropyl,
(4) —CH₂-cyclopropyl,
(5) phenyl, which is optionally substituted with from 1 to 3 substituents each of which is independently chloro, bromo, fluoro, —CN, —NO₂, —C₁₋₄ alkyl, or —O—C₁₋₄ alkyl,
(6) —CH₂-phenyl, where the phenyl is optionally substituted with from 1 to 3 substituents each of which is independently chloro, bromo, fluoro, —CN, —NO₂, —C₁₋₄ alkyl, or —O—C₁₋₄ alkyl,
(7)-HetA,
(8) —CH₂-HetA,
(9)-HetB, or
(10) —CH₂-HetB;
HetA is a heteroaromatic ring selected from the group consisting of pyrrolyl, pyrazolyl, imidazolyl, triazolyl, tetrazolyl, thiophenyl, thiazolyl, isothiazolyl, oxazolyl, isoxazolyl, oxadiazolyl, pyridinyl, pyridazinyl, pyrimidinyl, and pyrazinyl; wherein the heteroaromatic ring is optionally mono-substituted with a —C₁₋₄ alkyl, phenyl, or benzyl; and
HetB is a saturated heterocylic ring selected from the group consisting of pyrrolidinyl, pyrazolidinyl, imidazolidinyl, thiazolidinyl, isothiazolidinyl, oxazolidinyl, isoxazolidinyl, piperidinyl, piperazinyl, morpholinyl, and thiomorpholinyl; wherein the heterocyclic ring is optionally mono-substituted with a —C₁₋₄ alkyl, phenyl, or benzyl.

4. The process according to claim 1, wherein R² is:
(1) —C₁₋₆ alkyl,
(2) phenyl, which is optionally substituted with from 1 to 3 substituents each of which is independently halo, —CN, —NO₂, —CO₂H, —C(═O)—O—C₁₋₃ alkyl, —C₁₋₄ alkyl, or —O—C₁₋₁₄ alkyl, or
(3) —CH₂-phenyl, where the phenyl is optionally substituted with from 1 to 5 substituents each of which is independently halo, —CN, —NO₂, —CO₂H, —C(═O)—O—C₁₋₃ alkyl, —C₁₋₄ alkyl, or —O—C₁₋₄ alkyl.

5. The process according to claim 4, wherein R² is benzyl.

6. The process according to claim 1, wherein R³ is a branched —C₃₋₆ alkyl.

7. The process according to claim 6, wherein R³ is t-butyl.

8. The process according to claim 1, wherein R⁴ is methyl.

9. The process according to claim 1, wherein the reaction is conducted at a temperature in a range of from about −25 to about 100° C.

10. The process according to claim 1, wherein the organic solvent is a polar organic solvent selected from the group consisting of alcohols, ethers, and tertiary amides.

11. The process according to claim 10, wherein the organic solvent is a C₁₋₆ alkyl alcohol.

12. The process according to claim 1, wherein the base is selected from the group consisting of metal alkoxides, tertiary amines, and diazabicycloalkenes.

13. The process according to claim 1, wherein the dioxyfumarate of Formula III is employed in an amount of at least about 1 equivalent per equivalent of the amidine of Formula II.

14. The process according to claim 1, wherein the base is employed (i) in an amount of at least about 2 equivalents per equivalent of the amidine of Formula II when the free amidine is employed and (ii) in an amount of at least about 3 equivalents per equivalent of the amidine of Formula II when the amidine salt is employed.

15. A process for preparing a compound of Formula VI: 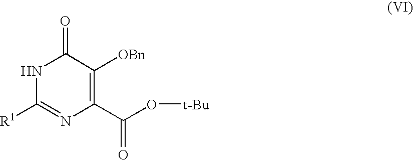
which comprises reacting an amidine of Formula II, or an acid salt thereof: 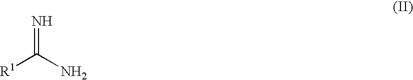
with a benzyloxyhydroxyfumarate of Formula Ia: 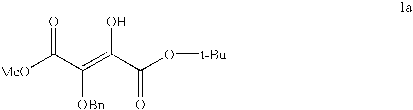
in an organic solvent and in the presence of a base to obtain the compound of Formula VI;
wherein R¹ is:
(1) —H,
(2) —O—C₁₋₄ alkyl,
(3) —CO₂R^{a},
(4) —C₁₋₄ alkyl,
(5) —C₁₋₄ alkylene-CO₂R^{a},
(6) —C₁₋₄ alkylene-N(R^{b}R^{c}),
(7) —C₃₋₆ cycloalkyl, which is optionally substituted with from 1 to 3 substituents each of which is independently a —C₁₋₄ alkyl,
(8)-C₁₋₄ alkylene-C₃₋₆ cycloalkyl, where the cycloalkyl is optionally substituted with from 1 to 3 substituents each of which is independently a —C₁₋₄ alkyl,
(9)-phenyl, which is optionally substituted with from 1 to 3 substituents each of which is independently halo, —CN, —NO₂, —CO₂R^{a}, —C₁₋₄ alkyl, or —O—C₁₋₄ alkyl,
(10) —C₁₋₄ alkylene-phenyl, where the phenyl is optionally substituted with from 1 to 3 substituents each of which is independently halo, —CN, —NO₂, —CO₂R^{a}, —C₁₋₁₄ alkyl, or —O—C₁₋₄ alkyl,
(11)-HetA,
(12) —C₁₋₄ alkylene-HetA,
(13)-HetB, or
(14)-C₁₋₄ alkylene-HetB;
R^{a} is —H or —C₁₋₄ alkyl;
R^{b} and R^{c} are each independently:
(1) —C₁₋₄ alkyl,
(2) allyl,
(3) phenyl, which is optionally substituted with from 1 to 3 substituents each of which is independently halo, —NO₂, —C₁₋₄ alkyl, or —O—C₁₋₄ alkyl,
(4) —CH₂-phenyl, where the phenyl is optionally substituted with from 1 to 3 substituents each of which is independently halo, —NO₂, —C₁₋₄ alkyl, or —O—C₁₋₄ alkyl,
(5) —C(═O)—O—C₁₋₄ alkyl, or
(6) —C(═O)—O—CH₂-phenyl, where the phenyl is optionally substituted with from 1 to 3 substituents each of which is independently halo, —NO₂, —C₁₋₄ alkyl, or —O—C₁₋₄ alkyl;
HetA is a 5- or 6-membered heteroaromatic ring containing from 1 to 4 heteroatoms independently selected from N, O and S; wherein the heteroaromatic ring is (i) optionally substituted with from 1 to 3 substituents each of which is independently halo or a —C₁₋₄ alkyl and (ii) optionally mono-substituted with phenyl or —(CH₂)₁₋₂-phenyl;
HetB is a 4- to 7-membered saturated heterocyclic ring containing at least one carbon atom and a total of from 1 to 4 heteroatoms independently selected from N, O and S; wherein any ring sulfur, if present, is optionally substituted with oxo or dioxo; and wherein the heterocyclic ring is (i) optionally substituted with from 1 to 0.3 substituents each of which is independently halo or a —C₁₋₄ alkyl and (ii) optionally mono-substituted with phenyl, —(CH₂)₁₋₂-phenyl, HetC, or —(CH₂)₁₋₂-phenyl-HetC; and
HetC is (i) a 5- or 6-membered heteroaromatic ring containing from 1 to 4 heteroatoms independently selected from N, O and S or (ii) a 4- to 7-membered saturated heterocyclic ring containing at least one carbon atom and from 1 to 4 heteroatoms independently selected from N, O and S.

16. The process according to claim 15, wherein R¹ is:
(1)—H,
(2) —C₁₋₄ alkyl,
(3) cyclopropyl,
(4) —CH₂-cyclopropyl,
(5) phenyl, which is optionally substituted with from 1 to 3 substituents each of which is independently chloro, bromo, fluoro, —CN, —NO₂, —C₁₋₁₄ alkyl, or —O—C₁₋₄ alkyl,
(6) —CH₂-phenyl, where the phenyl is optionally substituted with from 1 to 3 substituents each of which is independently chloro, bromo, fluoro, —CN, —NO₂, —C₁₋₄ alkyl, or —O—C₁₋₄ alkyl,
(7)-HetA,
(8)—CH₂-HetA,
(9)-HetB, or
(10) —CH₂-HetB;
HetA is a heteroaromatic ring selected from the group consisting of pyrrolyl, pyrazolyl, imidazolyl, triazolyl, tetrazolyl, thiophenyl, thiazolyl, isothiazolyl, oxazolyl, isoxazolyl, oxadiazolyl, pyridinyl, pyridazinyl, pyrimidinyl, and pyrazinyl; wherein the heteroaromatic ring is optionally mono-substituted with a —C₁₋₄ alkyl, phenyl, or benzyl;
HetB is a saturated heterocylic ring selected from the group consisting of pyrrolidinyl, pyrazolidinyl, imidazolidinyl, thiazolidinyl, isothiazolidinyl, oxazolidinyl, isoxazolidinyl, piperidinyl, piperazinyl, morpholinyl, and thiomorpholinyl; wherein the heterocyclic ring is optionally mono-substituted with a —C₁₋₄ alkyl, phenyl, or benzyl.

17. The process according to claim 15, wherein:
the organic solvent is a polar organic solvent selected from the group consisting of alcohols, ethers, and tertiary amides;
the base is selected from the group consisting of metal alkoxides, tertiary amines, and diazabicycloalkenes;
the benzyloxyhydroxyfumarate of Formula 1a is employed in an amount of at least about 1 equivalent per equivalent of the amidine of Formula II;
wherein the base is employed (i) in an amount of at least about 2 equivalents per equivalent of the amidine of Formula II when the free amidine is employed and (ii) in an amount of at least about 3 equivalents per equivalent of the amidine of Formula II when the amidine salt is employed; and
the reaction is conducted at a temperature in a range of from about −25 to about 100° C.

18. A compound of Formula III-A: 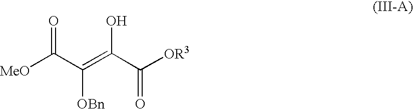
wherein R³ is —C₁₋₆ alkyl.

19. The compound according to claim 18, wherein R³ is a branched —C₃₋₆ alkyl.

20. The compound according to claim 19, wherein R³ is t-butyl.
